(19) United States
(12) Patent Application Publication
(10) Pub. No.: US 2006/0169031 A1
(43) Pub. Date: Aug. 3, 2006

(54) On-line monitoring of degredation and contamination of lubricant of rotating equipment

(76) Inventors: Limin Song, West Windsor, NJ (US); Michael F. Raterman, Doylestown, PA (US)

Correspondence Address: ExxonMobil Research and Engineering Company, P. O. Box 900, Annandale, NJ, 08801-0900, US

(21) Appl. No.: 11/312,295

(22) Filed: Dec. 20, 2005

Related U.S. Application Data
(60) Provisional application No. 60/645,700, filed on Jan. 21, 2005.

(51) Int. Cl. G01N 33/28

(52) U.S. Cl. 73/53.05; 702/25

(57) ABSTRACT

The present invention is a system for the determination of the useful life of a lubricant and detection of water or process fluid contamination level contained in a mechanical system. The system includes a lubricant sensor to measure a physical or chemical property of the lubricant, a database, a computer having access to the database, and a data acquisition system for providing power to the sensor and converting the measurement to numerical values for data storage. In a preferred embodiment the physical or chemical property is the dielectric constant.

This application claims the benefit of U.S. Provisional application 60/645,700 filed Jan. 21, 2005.

### BACKGROUND OF THE INVENTION

The present invention relates to a system for determining the useful life of a lubricant of a mechanical system. In particular, the system provides an on-line determination of useful life by measuring the dielectric constant.

A major root cause of machine bearing failures is the contamination of lubricants. The abnormal presence of contaminants such as moisture, dirt, and solid particle in the fluid causes the bearing wear (suction, seizure, erosion, and corrosion). Preventive maintenance is widely used in a process plant to keep the lubricant in good condition. This requires that the lubricant is changed on a fixed schedule (either time-based or usage based). This method is inefficient because the maintenance is performed based on a fixed schedule as opposed to lubricant's true condition.

The dominant method of the lubricant monitoring used in process plants is the lab analysis by which the lubricant is sampled periodically from the lubricant circuit in a pre-determined interval. The lubricant samples are either sent to a designated laboratory for analysis or analyzed by portable instrument.

On-line condition monitoring targets both the warning signs of impeding failure and the recognition of small failure that begins chain reaction that leads to big failures. The use of real-time vibration monitor has been effective at recognizing the symptoms of impeding machine failure and providing an early warning, from a few hours to a few days, which reduces the number of breakdowns and “catastrophic” failures. Though the real-time vibration monitoring could detect various machine problems, real-time monitoring of the lubricant are more effective for lubricant-related bearing problems since it measures the root cause directly and could provide longer lead time of predicting impeding bearing failure that allows optimal maintenance.

### SUMMARY OF THE INVENTION

The present invention is an on-line system to remotely monitor the condition of lubricants of mechanical equipment used in process plants and provide operation and maintenance personnel with timely diagnostic information on equipment. The system includes one or more lubricant sensors, a computer-based data acquisition system, a knowledge database, and a computer. The on-line system trends the normal aging of the lubricant for prediction of remaining useful life, identifies a correct lubricant to be used, detects abnormal contamination of the lubricant by processing fluid or water to avoid catastrophic equipment failure, and reduce the frequency for lubricant sampling and laboratory analysis. The system may be part of the plant network and the lubricant information on a piece of equipment could be accessed remotely by a number of users at the same time. The on-line system could also be integrated with other condition monitoring devices such as on-line vibration and bearing temperature monitors to give a more complete description of the equipment health condition and effective diagnosis. The on-line system could be applied to monitor compressors, turbine, pumps, motors, and other equipment that uses lubricant.

### BRIEF DESCRIPTION OF THE DRAWINGS

FIG. 1 is a schematic diagram of the present invention.

FIG. 2 shows water contamination of two lubricants.

FIG. 3 shows the relationship between dielectric constant and useful life of the lubricant.

### DETAILED DESCRIPTION OF THE PREFERRED EMBODIMENTS

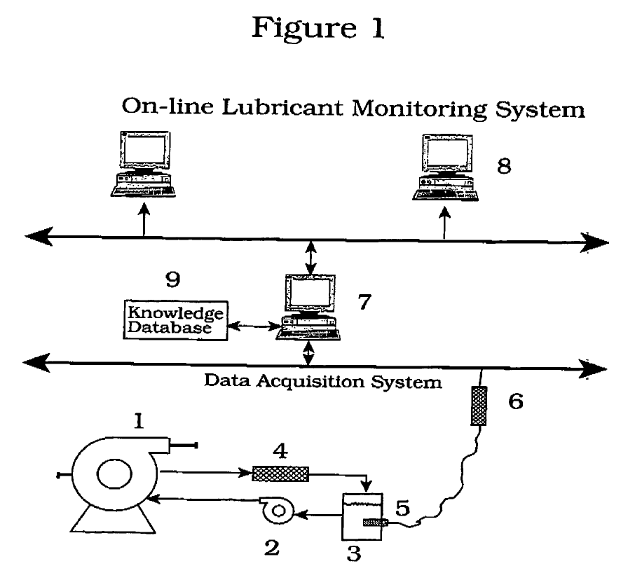

A schematic diagram of the on-line system of the present invention is shown in FIG. 1. The system includes a computer 7, a database 9, a sensor 5 for measuring an electro-chemical property of the lubricant. The computer and database are included in a data acquisition system for converting analogue signals from the sensor to digital signals for the computer. The sensor needs a source of power 6, which may be part of the sensor or part of the data acquisition system.

The sensor may be directly connected to the lubricant reservoir 3 of the mechanical equipment 1 or the lubricant may be circulated from the equipment to the sensor. In preferred embodiments, the system may also include a filter 4, a lubricant circulation device 2, and remote displays 8.

In a preferred embodiment, the lubricant sensor measures the dielectric constant of the lubricant and converts the measurement to a voltage signal. As the lubricant degrades due to friction, oxidation, temperature or contamination, the dielectric constant increases. The change in the dielectric constant of the lubricant represents the condition change in the lubricant. The temperature effect on the dielectric constant is compensated either by a built-in circuit in the sensor or an algorithm in the computer so that the output of the sensor represents the sole effect of the lubricant degradation over the base.

Through the plant network, the data from the data acquisition system can be accessed remotely by different user at the same time. The lubricant data can also be correlated with other on-line monitoring data such as bearing house vibration and temperature on the same machine for diagnosis of more complicated machine problems.

The knowledge database consists of calibration data for a specific lubricant, and a computerized data analysis. The typical calibration data include:

1) Typical initial value of the sensor output when a fresh lubricant is put into the machine system and its statistical bounds.

2) Typical end value of the sensor output when the lubricant is no longer useful and needs to be changed and its statistical bounds.

3) Normal aging curve of the lubricant against normal usage time and its statistical bounds.

4) Typical sensitivity of the water contamination level detection and its statistical bounds.

The computerized data analysis system in the knowledge database compares the measurement and calibration data and provides the inference on the condition of the lubricant based on the current and historical sensor data and actionable recommendation to operation and maintenance personnel. The target applications of the on-line lubricant monitoring system include:

1) Identify right lubricant is put into the machine system based on the fact that different type of lubricants have different dielectric constant values.

2) Estimate water moisture level based on the correlation of the sensor output with the water moisture level.

3) Detect bulk or insolvable water or process fluid contamination based on the presence of the spikes in sensor output

4) Forecast remaining useful life of the lubricant based on current sensor output against normal aging curve.

As an example of the first application, the following table shows the outputs of the on-line monitoring system for two different fresh lubricants for rotating equipment (Type I and Type II for reference herein):

| | Average | Standard |
|---|---|---|
| | Sensor Output | Deviation |
| Type I Lubricant | 0.373 V | 1.9 mV |
| Type II Lubricant | 1.308 V | 1.4 mV |

The significant difference between two types of the lubricants can be used to identify that the right type of the lubricant is place for a specific machine system.

The detection of water contamination in the machine lubricant system is very important for lubricant monitoring of large rotating equipment in process plants. The presence of the water in the lubricant will reduce the lubricant properties, which may be sufficient to increase the friction between the moving parts and cause excessive wear. In addition, the presence of water over a prolonged period can cause corrosion. The literature shows that the bearing life can be dramatically extended when the moisture is controlled below 500 PPM.

There are two types of water or process fluid contamination that could occur in the lubrication circulation system. One is the presence of water in emulsion state or moisture in the lubricant; another is the presence of water in bulk or insolvable state. The accumulation of the moisture would gradually increase the dielectric constant of the mixture and the leaking of water or process fluid into lubricant would cause spikes or sudden change in the sensor output because of significant difference in dielectric constant between the lubricant and water or process fluid.

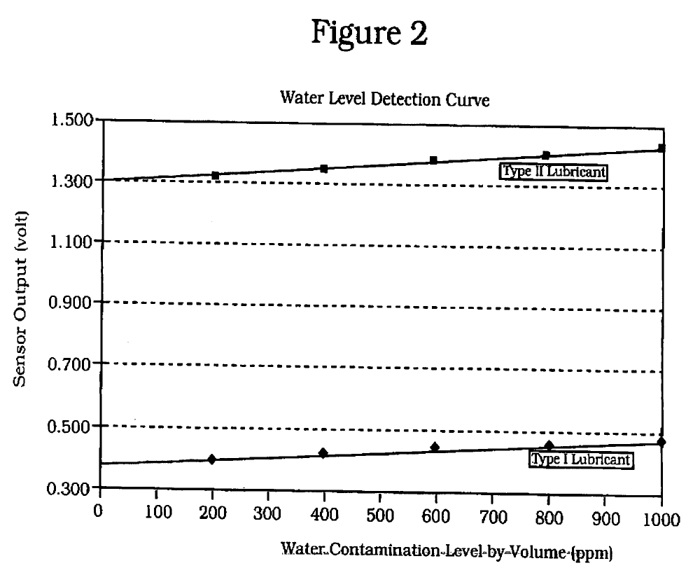

FIG. 2 presents the water detection curves obtained from the testing of two types of the lubricants (Type I and Type II). The correlation of the output of the on-line monitoring system with the water level (PPM by volume) is nearly linear and almost identical except that the different offset at the zero water levels for different lubricants. If the lubricant is contaminated by water moisture, the change in the sensor output and water detection curve can be used to infer the water contamination level. Since the normal aging of the lubricant also increases the dielectric constant, the value of the normal aging curve, to be discussed next, will be subtracted from the sensor output before the water detection curve is used for water level inference. Detection of high level water level by the sensor can be used to alert machine operator for either purification or change of oil.

If the bulk water or process fluid is present, the amplitude of the spikes and frequency of the spike occurrence in the sensor output can be used to detect the severity of the contamination. Locating the sensor closer to where leak is suspected is preferred to detect bulk water or process fluid contamination.

Additional benefit of the on-line lubricant monitoring system is its capability of forecasting remaining useful life of the lubricant. Knowing the remaining useful life of the lubricant, equipment operation and maintenance personnel could perform the lubricant maintenance based on actual condition instead of the “fixed” interval, which would maximize the use of the lubricant and reduce the number of the sampling for lab analysis.

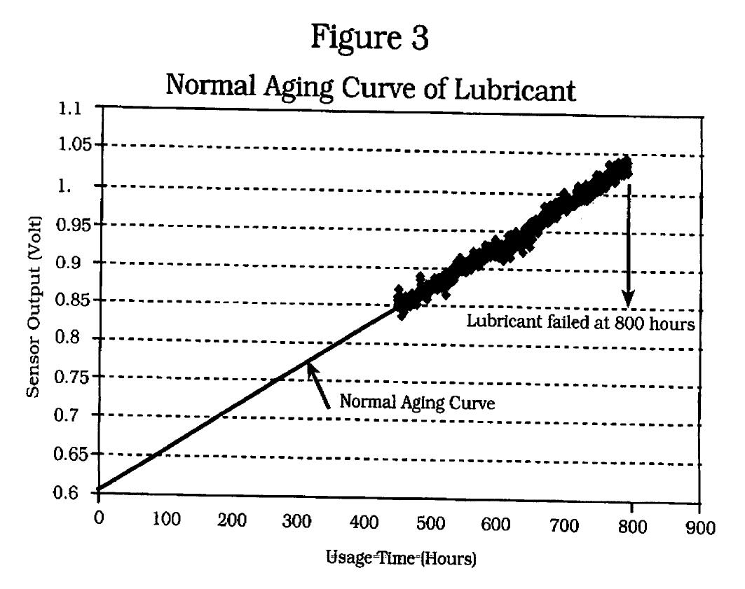

Forecasting the remaining useful life is based on the linear relationship between the dielectric constant and the usage time of the lubricant. This linear relationship is demonstrated in FIG. 3, which presents the testing data on a lubricant oil which was tested in a pump system until it was determined failed. The linear relationship, or normal aging curve, provides a simple way to forecast the remaining useful life of the lubricant.
tᵣ={(Vₑ Vₒ)/(V−Vₒ)−1.0}t
where
tr—remaining useful life of the lubricant at usage time of t
V—average value of the sensor output at usage time of t
Vₒ—average value of the sensor output when a fresh lubricant is put into the machine system
Vₑ—average value of the sensor output when the lubricant is no longer useful and need to be changed
   To establish the normal aging curve requires the values of both Vₒ and Vₑ. The value of Vₒ can be determined from the dielectric sensor either in laboratory or in-situ when the lubricant is replaced by fresh oil. The value of Vₑ can be determined from the dielectric sensor by measuring the dielectric value of final oil either in the lab or in-situ.

What is claimed is:

1. A system for the determination of the useful life of a lubricant and/or detection of water or process fluid contamination level contained in a mechanical system comprising
(a) a lubricant sensor to measure a physical or chemical property of the lubricant,
(b) a database,
(c) a computer having access to the database,
(d) an on-line data acquisition system for converting the measurement to numerical values for storage in said database.

2. The system of claim 1 further comprising a circulation system for circulating lubricant from the mechanical system to the lubricant sensor.

3. The system of claim 2 wherein said circulation system includes a pump, and a means for bringing the lubricant into contact with the lubricant sensor.

4. The system of claim 3 wherein the circulation system further comprises a filter and a lubricant reservoir for contacting the lubricant with the sensor.

5. The system of claim 1 wherein the sensor is in contact with the mechanical system.

6. The system of claim 1 wherein said system is on-line with the mechanical system.

7. The system of claim 1 wherein said database includes data regarding the lubricant and the measured physical or chemical property in order to determine the remaining useful life of the lubricant.

8. The system of claim 1 wherein said physical or chemical property is dielectric constant.

9. The system of claim 1 wherein said sensor includes a circuit that compensates for the temperature effect of the physical or chemical property.
